(19) United States
(12) Patent Application Publication
(10) Pub. No.: US 2022/0172832 A1
(43) Pub. Date: Jun. 2, 2022

(54) ASSISTANCE SYSTEM

(71) Applicant: FUJI CORPORATION, Aichi-ken (JP)

(72) Inventors: Yuki KAMIYA, Toyoake-shi, Aichi-ken (JP); Takehiro HIRAOKA, Chiryu-shi, Aichi-ken (JP); Satoshi SHIMIZU, Chiryu-shi, Aichi-ken (JP); Ryu TAKAHASHI, Toyota-shi, Aichi-ken (JP)

(73) Assignee: FUJI CORPORATION, Aichi-ken (JP)

(21) Appl. No.: 17/436,068

(22) PCT Filed: Mar. 5, 2019
(86) PCT No.: PCT/JP2019/008718

§ 371 Date: Sep. 2, 2021

(51) Int. Cl. G16H 40/63; A61G 7/10

(52) U.S. Cl. CPC G16H 40/63; A61G 2203/20; A61G 7/1019; A61G 7/1007

(57) ABSTRACT

An assistance system includes a storage section configured to store usage record data in which usage information as to a use of an assistance device configured to assist an operation of a care receiver is recorded in association with time information as to a date and time when the assistance device is used and user identification information used to identify a user using the assistance device, an extracting section configured to extract the usage information and the time information as to a specific user based on the user identification information included in the usage record data, and a display section configured to display a time series change in the usage information as to the specific user in a graspable manner.

### TECHNICAL FIELD

The present invention relates to an assistance system.

### BACKGROUND ART

Patent Literature 1 discloses an assistance device configured to assist a transfer operation of a care receiver. In assisting the transfer operation, the assistance device performs a directional change after completion of a standing operation of lifting the buttocks of the care receiver in a sitting posture from a seat surface and performs a sitting operation of lowering the buttocks of the care receiver to sit the care receiver. As a result, the care receiver is transferred from a bed to a wheelchair, for example.

### PATENT LITERATURE

Patent Literature 1: JP-A-2008-073501

### SUMMARY

### Technical Problem

In a facility or the like where an assistance device is to be introduced, it is required that a caregiver or the like assisting a care receiver detect a change in a physical condition of the care receiver, who is a user of the assistance device, at an early stage. In this regard, there is a demand to predict the change in the physical condition of the care receiver based on changes in a use status of an assistance device.

It is an object of the present specification to provide an assistance system capable of predicting the change in the physical condition of the care receiver by grasping changes in a use status of the assistance device by the care receiver.

### Solution to Problem

Firstly, the present specification discloses an assistance system including a storage section configured to store usage record data in which usage information as to a use of an assistance device configured to assist an operation of a care receiver is recorded in association with time information as to a date and time when the assistance device is used and user identification information used to identify a user using the assistance device, an extracting section configured to extract the usage information and the time information as to a specific user based on the user identification information included in the usage record data, and a display section configured to display a time series change of the usage information as to the specific user in a graspable manner.

Secondly, the present specification discloses an assistance system including a storage section configured to store usage record data in which usage information as to a use of an assistance device configured to assist an operation of a care receiver is recorded in association with time information as to a date and time when the assistance device is used and user identification information used to identify a user using the assistance device, an extracting section configured to extract the usage information and the time information as to a specific user based on the user identification information included in the usage record data, and a display section configured to display an analysis result as to a change in a physical condition of the user based on a time series change in the usage information as to the specific user.

### Advantageous Effect of the Invention

According to the first disclosure, the display section displays the time series change in the usage information as to the specific user in a graspable manner. As a result, the caregiver or the like assisting the care receiver can grasp changes in a use status of the assistance device by the care receiver. Then, the caregiver or the like can predict the change in the physical condition of the care receiver based on the changes in the use status of the assistance device by the care receiver.

According to the second disclosure, the display section displays the analysis result as to the change in the physical condition of the specific user based on the time series change in the usage information as to the specific user. As a result, the caregiver or the like assisting the care receiver can grasp changes in a use status of the assistance device by the care receiver. Then, the caregiver or the like can predict the change in the physical condition of the care receiver based on the analysis result as to the change in the physical condition of the specific user.

### BRIEF DESCRIPTION OF DRAWINGS

FIG. 1 is a schematic view of an assistance system according to an embodiment.

FIG. 2 is a side view of an assistance device for supporting a care receiver in a sitting posture.

FIG. 3 is a side view of an assistance device for supporting a care receiver in a ready-to-stand posture.

FIG. 4 is a side view of an assistance device for supporting a care receiver in a stand-up posture.

FIG. 5 illustrates a configuration of a management device.

FIG. 6 illustrates an example of usage record data.

FIG. 7 illustrates an example of assistance record data.

FIG. 8 illustrates an example of care receiver data.

FIG. 9 is a graph illustrating a transition of the number of uses of the assistance device.

FIG. 10 is a graph illustrating a transition of specific load value information.

FIG. 11 is a graph illustrating a transition of load fluctuation information.

FIG. 12 is a graph illustrating a transition of the number of uses of the assistance device and a transition of the number of assistance records by action content.

FIG. 13 is a graph illustrating usage information, application information, and assistance record information of the assistance device per day.

### DESCRIPTION OF EMBODIMENTS

### 1. Overview of Assistance System

An assistance system records, as usage record data, usage information obtained during use of an assistance device configured to assist an operation of a care receiver. In the usage record data, user identification information capable of identifying a user who used the assistance device (i.e., care receiver) is recorded in association with the usage information. Accordingly, the assistance system can extract the usage record data of a specific user. Further, in the usage record data, usage time information as to a date and time when the assistance device is used is stored in association with the usage information. As a result, the assistance system can grasp a time series change in the usage information as to the specific user. Then, the assistance system presents the time series change in the usage information, or an analysis result as to a change in a physical condition of a user predicted based on the time series change in the usage information.

As a result, a caregiver or the like assisting the care receiver who is the user can predict a deterioration of the physical condition of the care receiver who is the user based on the time series change in the usage information presented by the assistance system or the analysis result as to the change in the physical condition of the user. In addition, the caregiver, a developer of the assistance device, and the like can perform an effect measurement by use of the assistance device based on the time-series change in the usage information presented by the assistance system or the analysis result as to the change in the physical condition of the user.

### 2. Schematic Configuration of Assistance System 1

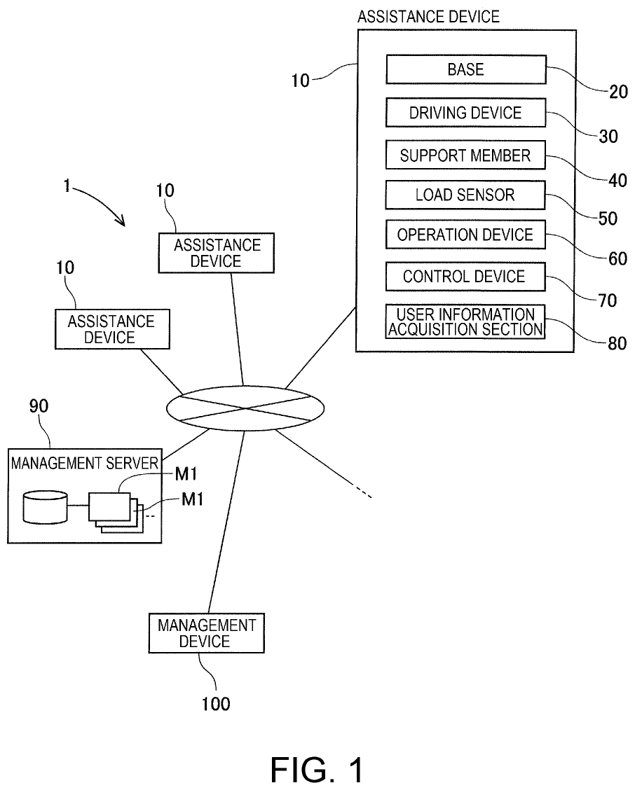

Embodiments of the assistance system will be described below with reference to the accompanying drawings. First, a schematic configuration of assistance system 1 will be described with reference to FIG. 1.

As illustrated in FIG. 1, assistance system 1 mainly includes one or more assistance devices 10, management server 90, and one or more management devices 100. Assistance device 10 is used when assisting the operation of the care receiver. In assistance system 1, all assistance devices 10 and management device 100 are communicably connected to management server 90 via the Internet, and management device 100 integrates and manages all assistance devices 10 provided in assistance system 1.

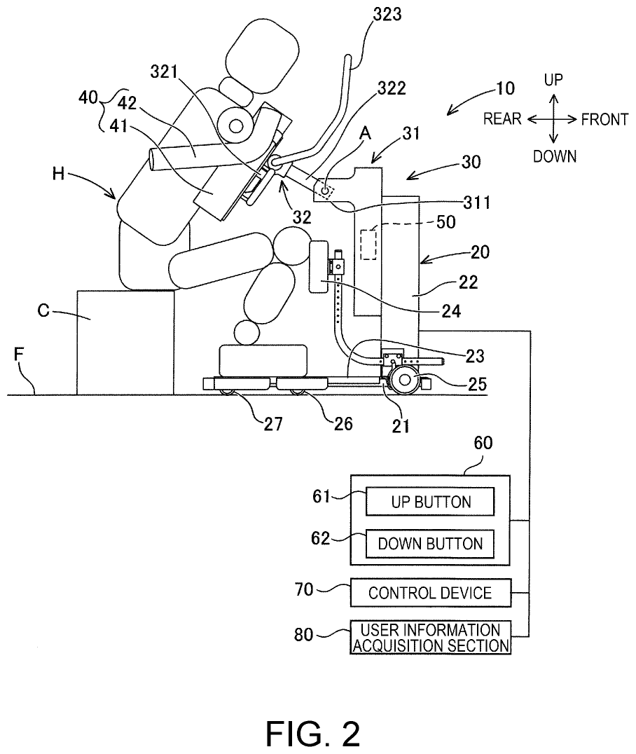

Assistance device 10 assists a standing operation of user H (refer to FIG. 2) from a sitting posture to a stand-up posture, and assists a sitting operation from the stand-up posture to the sitting posture. It should be noted that the “stand-up posture” of user H means a posture in which a lower body of user H is upright, regardless of the posture of an upper body. That is, the standing operation of user H is an operation for lifting the buttocks of user H to the stand-up posture. The sitting operation of user H is an operation for lowering the buttocks of user H to the sitting posture.

Assistance device 10 supports, for example, a portion of the body of user H (e.g., the upper body of user H), changes a direction after assisting the standing operation of user H in the sitting posture, and assists a transfer operation to assist the sitting operation so as to be sat again in another position. Such a transfer operation is performed, for example, for the purpose of transferring between a bed and a wheelchair in a living room, or between a bed in a living room and a toilet bowl in a toilet room.

In addition, assistance device 10 records various information obtained when assisting the operation of user H. Then, assistance device 10 uploads the recorded various information to management server 90 as usage record data M1.

Management server 90 stores usage record data M1 uploaded from multiple assistance devices 10. Then, management server 90 transmits usage record data M1 to management device 100 in response to a request of management device 100. Here, management server 90 can transmit only a part of usage record data M1 corresponding to the request among all stored usage record data M1 to management device 100.

For example, management server 90 may transmit only usage record data M1 as to specific user H to management device 100. In addition, when multiple types of usage information are included in usage record data M1, management server 90 can transmit only a specific type of usage information to management device 100. As a result, management device 100 can appropriately download only necessary information from management server 90.

Management device 100 treats as management targets all assistance devices 10 provided in assistance system 1, and acquires usage record data M1 recorded by each of assistance devices 10 from management server 90. Then, management device 100 provides various types of information based on the acquired usage record data M1. Examples of management device 100 include a personal computer installed in a facility where assistance device 10 is installed, and a terminal device such as a portable terminal (smart phone, tablet terminal), for example. In addition, management device 100 can add information acquired from the outside of assistance device 10 to usage record data M1 acquired from assistance device 10 via management server 90.

### 3. Configuration of Assistance Device 10

Next, a configuration of assistance device 10 will be described with reference to FIG. 2. As illustrated in FIG. 2, assistance device 10 mainly includes base 20, driving device 30, support member 40, load sensor 50, operation device 60, control device 70, and user information acquisition section 80.

Base 20 mainly includes frame 21, support column 22, footrest 23, lower limb contacting section 24, and six wheels 25 to 27. Frame 21 is provided substantially horizontally near floor F. Support column 22 is fixed to frame 21 while extending upward from the center of a left-right direction at the front of frame 21. Support column 22 may be provided so as to be perpendicular to floor F or at a predetermined angle in a front-rear direction.

Footrest 23 is fixed to the rear of the upper face of frame 21 so as to be horizontal. Lower limb contacting section 24 is fixed to support column 22 so as to be positioned above footrest 23. Lower limb contacting section 24 has a cushion member at a portion with which the lower limb of care receiver H comes into contact. On a lower side of frame 21, three wheels 25 to 27 are provided to both the left and right sides. Each of wheels 25 to 27 has a steering function for changing a movement direction. With the steering function of six wheels 25 to 27, assistance device 10 is not only capable of moving and changing directions in the front-rear direction, but also moving laterally (moving directly to the side) and spin turn (i.e., rotating on the spot).

Driving device 30 supports support member 40 that supports the upper body of user H so as to be movable in a vertical direction and the front-rear direction of base 20. Driving device 30 includes lifting and lowering section 31 and swing section 32. Driving device 30 is controlled by control device 70 to operate lifting and lowering section 31 and swing section 32. Driving device 30 is configured to move support member 40 along a predetermined movement locus by coordination of vertical movement of lifting and lowering section 31 and rotational movement of swing section 32.

Lifting and lowering section 31 moves linearly in the vertical direction with respect to base 20. Lifting and lowering section 31 has an elongated shape extended in the vertical direction and is guided by a guide (not shown) on a rear surface of support column 22. Lifting and lowering section 31 is lifted and lowered along the guide of support column 22 by driving of a linear motion device (not shown). Inside lifting and lowering section 31, a motor (not shown) for rotating swing section 32 is accommodated. Lifting and lowering section 31 has swing support section 311. Swing support section 311 rotatably supports swing section 32 around center shaft A.

Swing section 32 rotates around center shaft A provided in lifting and lowering section 31 and causes support member 40 to swing. Swing section 32 includes swing main body 321, arm 322, and handle 323. Swing main body 321 is an attachment section to which support member 40 is attached and detached, and arm 322 and handle 323 are integrally fixed to swing main body 321.

One end of arm 322 is rotatably supported around center shaft A of swing support section 311 of lifting and lowering section 31. Handle 323 is formed in a substantially rectangular frame shape. Arm 322 is rotated by the driving of the motor. When assistance device 10 assists the standing operation, arm 322 is mainly rotated forward from a state extending rearward. On the other hand, when assistance device 10 assists the sitting operation, arm 322 is mainly rotated rearward so as to extend rearward. With the above configuration, swing section 32 rotates around a horizontal shaft (center shaft A) parallel to the left-right direction of base 20 and swings support member 40 attached to swing main body 321 at the distal end of arm 322.

Handle 323 is formed so as to extend in a front-upper direction from a vicinity of a rear end of arm 322. A side portion of handle 323 is used as a portion gripped by both hands of user H. In addition, side and front portions of handle 323 are used as portions to be gripped by the caregiver to move assistance device 10.

Support member 40 is a member that supports the upper body of user H. Support member 40 includes trunk supporting section 41 and a pair of underarm supporting sections 42. trunk supporting section 41 is formed in a surface shape that closely matches the trunk shape of user H, so that it can be flexibly deformed. The support surface of trunk supporting section 41 is contact with a front surface of the trunk of the upper body of user H to support the trunk. Specifically, the support surface of trunk supporting section 41 supports a range from the chest to the abdomen of user H from below. Further, trunk supporting section 41 is attached to swing main body 321.

The pair of underarm supporting sections 42 is supported by trunk supporting section 41, supports the underarm of user H. Specifically, the pair of underarm supporting sections 42 is supported so as to be swingable relative to trunk supporting section 41 on both sides in the left-right direction of trunk supporting section 41. Underarm supporting section 42 is a rod-like member having an L-shape. The surface of underarm supporting section 42 is covered by a material that can be flexibly deformed.

Load sensor 50 is a load cell capable of detecting a load received by assistance device 10. Specifically, load sensor 50 is provided in lifting and lowering section 31. That is, lifting and lowering section 31 is a portion that holds the upper body of user H during an operation of assistance device 10, and load sensor 50 detects a downward load that lifting and lowering section 31 receives from user H during the operation of assistance device 10. As a result, assistance device 10 can grasp the load applied to assistance device 10 from the upper body of user H. It should be noted that a detection value detected by load sensor 50 is output to control device 70 as a load value received by assistance device 10.

Here, the present embodiment will be described using a case where assistance device 10 includes one load sensor 50, but it is also possible for assistance device 10 to include multiple load sensors 50. For example, assistance device 10 may provide lifting and lowering section 31 with load sensor 50 for detecting a forward load applied from user H by lifting and lowering section 31 during the operation of assistance device 10. As a result, assistance device 10 can grasp the forward load received by assistance device 10 from user H.

In addition, assistance device 10 may provide load sensor 50 to lower limb contacting section 24. In this case, assistance device 10 can detect the load applied to assistance device 10 from the lower limb of user H. For example, in a case where assistance device 10 is configured to support the lower limb of user H in the stand-up posture by lower limb contacting section 24, it is possible to grasp the forward load received by assistance device 10 from user H.

In addition, assistance device 10 may provide load sensor 50 on footrest 23. In this case, assistance device 10 can detect the downward load applied to assistance device 10 from the lower body of user H. In addition, assistance device 10 can grasp a ratio of the load applied from the upper body of user H to the load applied from the lower body of user H, during the operation of assistance device 10, by grasping both the downward load received by lifting and lowering section 31 and the downward load received by footrest 23.

Operation device 60 has multiple buttons corresponding to various operations assisted by assistance device 10. Operation device 60 includes up button 61 corresponding to the up operation and down button 62 corresponding to the lowering operation. Operation device 60 is connected to control device 70 via, for example, an extendable signal cable. When any button is pressed, operation device 60 transmits to control device 70 a signal corresponding to the type of the button for a period of time the button is pressed.

Control device 70 controls the operations of lifting and lowering section 31 and swing section 32 of driving device 30. In the operation processing for assisting the standing operation or the sitting operation of user H, control device 70 activates lifting and lowering section 31 and swing section 32 based on the operation for operation device 60. Control device 70 controls the movement of support member 40 in cooperation with the vertical movement of lifting and lowering section 31 and the rotational movement of swing section 32 when performing the standing operation and the sitting operation.

In addition to the above, control device 70 records the information acquired during the operation of assistance device 10. Specifically, control device 70 records usage time information as to a date and time when assistance device 10 is used, and usage information as to a usage of assistance device 10. Examples of the usage time information include, for example, a date and time when a signal from operation device 60 is received, a date and time when assistance device 10 during operation is placed in a specific posture set in advance. Examples of the usage information include load value information as to the load value detected by load sensor 50.

User information acquisition section 80 acquires user identification information capable of identifying user H who uses assistance device 10, and sends the acquired user identification information to control device 70. Examples of the method of acquiring the user identification information by user information acquisition section 80 include a method using a keyboard, a mouse, a touch panel, a camera, a microphone, and the like. Examples of the user identification information include ID information assigned to each care receiver who is user H, a voice of the care receiver, a face image, and the like.

For example, the care receiver who is user H has ID information as the user identification information, and user information acquisition section 80 acquires ID information inputted using an input device such as a keyboard, a mouse, and a touch panel. In addition, user information acquisition section 80 can display the care receiver registered in advance as user H on the touch panel or the like, and acquire the user identification information of one user H selected by the caregiver or the like from among displayed users H. In addition, user information acquisition section 80 can also acquire the user identification information of the specified user H by performing biometric authentication such as voice authentication based on a voice of user H acquired using the microphone, face authentication based on the face image of user H acquired using the camera, or the like.

### 4. Assisting Operation by Assistance Device 10

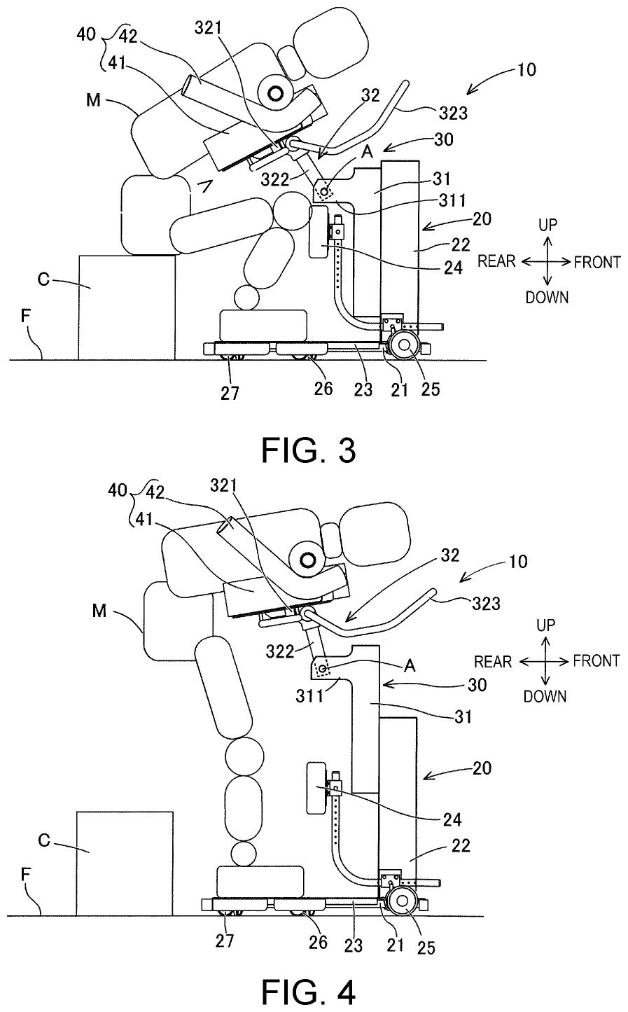

Next, a standing assistance operation of assistance device 10 will be described with reference to FIGS. 2 to 4. In the standing assistance operation, assistance device 10 enters a ready-to-stand preparation state illustrated in FIG. 3 from a starting state of the standing assistance operation illustrated in FIG. 2, and then enters a standing completion state illustrated in FIG. 4.

As illustrated in FIG. 2, a caregiver moves assistance device 10 to a vicinity of user H (care receiver) in a sitting posture. At this time, the caregiver activates assistance device 10 to bring a state such that user H in the sitting posture can board. In addition, the caregiver adjusts a height of lifting and lowering section 31 in accordance with the height of user H. Subsequently, user H causes both legs to enter the lower side of support member 40.

Next, user H places both feet on footrest 23, and causes the lower limb to contact lower limb contacting section 24. In addition, user H places a trunk on trunk supporting section 41. At this time, the upper body of user H is placed in a posture inclined slightly ahead in a state supported by support member 40. At the same time, user H causes underarm supporting section 42 to be inserted into the underarm. In this manner, assistance device 10 is set to the starting state of the standing assistance operation. Then, the caregiver causes user H to grip handle 323. The posture of user H at this time is a starting posture of the standing assistance operation. Subsequently, the caregiver initiates driving of assistance device 10 based on a standing assistance program of assistance device 10. As a result, lifting and lowering of lifting and lowering section 31 and forward tilting of swing section 32 are performed in a coordinated manner.

By executing the standing assistance program, assistance device 10 enters the ready-to-stand state illustrated in FIG. 3. The ready-to-stand state of assistance device 10 is a state immediately before user H in the sitting posture is lifted from chair C. That is, assistance device 10 enters the ready-to-stand state illustrated in FIG. 3 through lowering of lifting and lowering section 31 from the starting state illustrated in FIG. 2, and tilting ahead of swing section 32. Here, when assistance device 10 is placed in the ready-to-stand state, user H enters a state in which the trunk is tilted forward and extended in a state in which the buttocks are in contact with the seat surface of chair C. The posture of user H at this time is the ready-to-stand posture.

As illustrated in FIG. 4, when the standing assistance program is further continued, lifting and lowering section 31 is lifted and swing section 32 is further tilted ahead. As a result, user H enters the stand-up posture from the ready-to-stand posture. That is, the upper body of user H in the stand-up posture is placed in a posture greatly inclined ahead, and the position of the buttocks of user H is located at a position higher than the seat surface of chair C. Then, the leg section of user H is in a substantially extended state.

As described above, by tilting trunk supporting section 41 forward with user H boarded in assistance device 10, user H is transitioned from the starting posture in the sitting posture to the stand-up posture via the ready-to-stand posture. In addition, the operation of the seat assistance of assistance device 10 is performed in a substantially opposite manner to the operation of the standing assistance. That is, by lowering lifting and lowering section 31 while trunk supporting section 41 is tilted rearward, user H can be shifted from the stand-up posture to the sitting posture. Then, user H in the sitting posture can easily remove underarm supporting section 42 from the underarm.

The stand-up posture is a posture in which the load on the lower body of user H is increased. Accordingly, if the load received by assistance device 10 from the upper body of user H, who is in the stand-up posture, tends to increase, there is a fear that a physical condition deterioration such as a decrease in leg force is occurring in the care receiver who is user H. That is, the caregiver or the like can predict a change in the physical condition of the care receiver who is user H by grasping a fluctuation in the load received by assistance device 10 from the upper body of user H.

In addition, assistance device 10 uploads the usage information and the usage time information recorded by control device 70 and the user identification information acquired by user information acquisition section 80 to management server 90 as usage record data M1. At this time, the usage information is recorded in usage record data M1 in association with the user identification information and the usage time information.

### 5. Configuration of Management Device 100

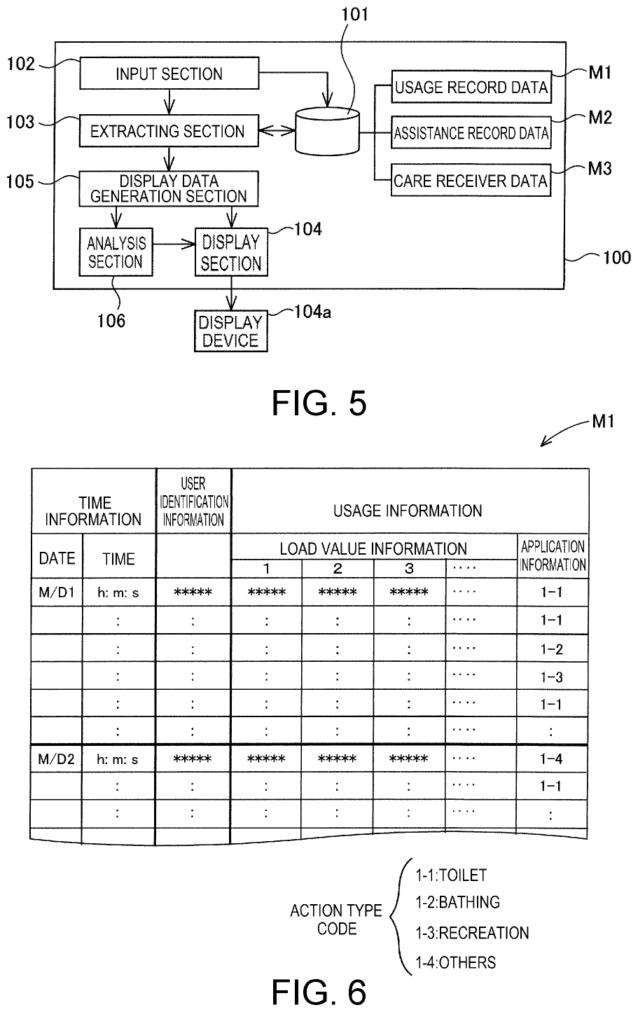

Next, a configuration of management device 100 will be described with reference to FIG. 5. As illustrated in FIG. 5, management device 100 mainly includes storage section 101, input section 102, extracting section 103, display section 104, display data generation section 105, and analysis section 106.

Storage section 101 is configured by an optical drive device such as a hard disk device, a flash memory, or the like. Storage section 101 stores usage record data M1 acquired from assistance device 10 via management server 90. In addition, usage record data M1 stores, as one of the usage information, application information acquired from the outside of assistance device 10 and capable of grasping an application of assistance device 10.

In addition, storage section 101 stores assistance record data M2 as to an assistance record performed for the care receiver who is user H. In assistance record data M2, assistance record information as to an assistance performed without using assistance device 10 is recorded in association with assistance time information as to a date and time when the assistance was performed and care receiver identification information as to the care receiver to whom the assistance was performed. The care receiver identification information includes, for example, the name of the care receiver, ID information assigned to each care receiver, or the like. In addition, storage section 101 stores care receiver data M3 used to associate the care receiver identification information of the care receiver registered as user H of assistance device 10 with the user identification information.

Input section 102 receives an input to an input device (not illustrated) operable by a caregiver or the like utilizing assistance system 1. The input device provided in management device 100 is, for example, a keyboard, a mouse, and a touch panel or the like. For example, when extracting the information as to a specific care receiver from various data stored in storage section 101, the caregiver or the like inputs the care receiver identification information of the care receiver. It should be noted that the input device can also be used when registering a care receiver to be a new user of assistance device 10 in care receiver data M3, or when adding a new assistance record to assistance record data M2.

When input section 102 receives the care receiver identification information, extracting section 103 extracts the user identification information associated with the care receiver identification information. Then, extracting section 103 extracts the usage information and the usage time information associated with the user identification information from usage record data M1, and extracts the assistance record information and the assistance time information associated with the care receiver identification information from assistance record data M2.

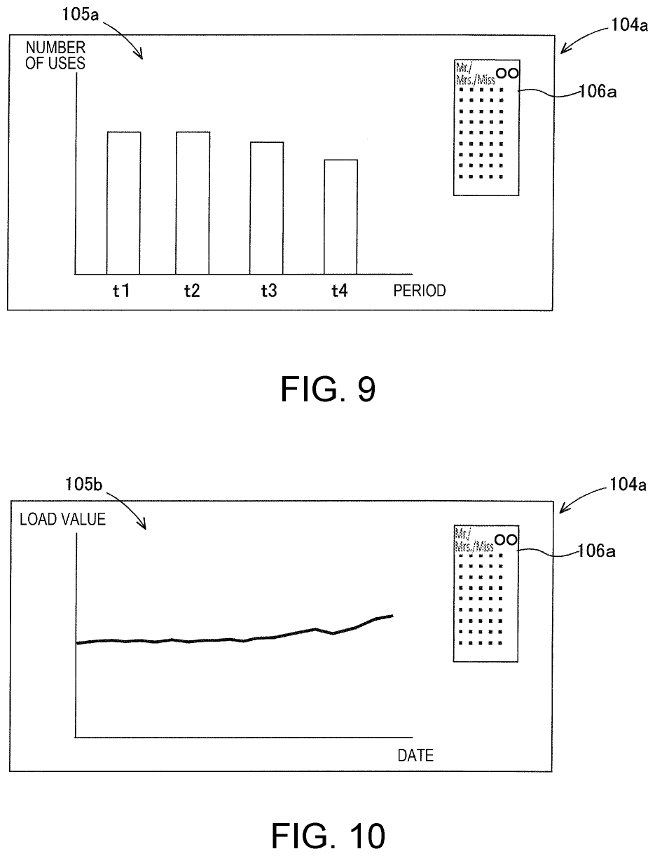

Display section 104 displays, on display device 104a (refer to FIG. 9), information as to a specific care receiver extracted by extracting section 103, and usage information when the care receiver uses assistance device 10 as user H. At this time, display section 104 displays the time series change in the usage information as to user H in a manner confirmed at a glance.

Display data generating section 105 generates display data displayed by display section 104 as data indicating the time series change of the usage information. The caregiver or the like can use the display data as data indicating a sign of a change in the physical condition of specific user H. The display data will be described later with specific examples.

Analysis section 106 performs analysis concerning the change in the physical condition of user H based on the time series change in the usage information as to specific user H extracted by extracting section 103. Then, display section 104 displays an analysis result by analysis section 106 on display device 104a (refer to FIG. 9).

### 6. Specific Example of Usage Record Data M1

Next, a specific example of usage record data M1 will be described with reference to FIG. 6. Usage record data M1 illustrated in FIG. 6 includes usage time information, user identification information, and usage information. In usage record data M1, the usage information is recorded in association with the usage time information and the user identification information.

In an example illustrated in FIG. 6, the usage time information is usage date and time information as to the date and time when the user identification information is acquired by user information acquisition section 80. It should be noted that usage record data M1 can also record, as the usage time information, the date and time when the operation with respect to operation device 60 is performed, and the date and time when assistance device 10 is placed in a predetermined posture (e.g., the posture of assistance device 10 when user H is placed in the stand-up posture). In addition, assistance device 10 acquires the usage time information at multiple different timings during a series of operations of assistance device 10, and usage record data M1 can record the usage period information calculated based on the multiple usage time information acquired during the series of operations of assistance device 10 as one of the usage time information. Examples of the use period information include a duration time of the stand-up posture, a time required until the transition from the ready-to-stand state to the standing completion state, and the like.

As described above, the user identification information is information associated with the care receiver identification information. In the example illustrated in FIG. 6, ID information assigned to each of the care receivers expected as user H is recorded as the user identification information.

The usage information is information obtained when assistance device 10 is operated. In the example illustrated in FIG. 6, usage record data M1 includes, as usage information, load value information as to the load value received by assistance device 10 when assistance device 10 is used, and application information of assistance device 10 acquired from the outside of assistance device 10.

The load value information is a load value detected by load sensor 50 during the operation of assistance device 10. In the example illustrated in FIG. 6, multiple pieces of load value information are recorded in usage record data M1. The multiple pieces of load value information are the information as to the load value detected by load sensor 50 at different timings during the series of operations of assistance device 10. Examples of the information included in the multiple load value information include: load value information received when assistance device 10 in operation assumes a specific posture; load value information detected every predetermined time period from the ready-to-stand state to the standing completion state; load value acquired every predetermined time period from the standing completion state; and the like. In addition, usage record data M1 can record as the load value information, a maximum value or an average value of the load value detected by load sensor 50 during the series of operations.

In addition, usage record data M1 can record load fluctuation information as to a fluctuation of the load value received by assistance device 10 during the operation of assistance device 10. For example, usage record data M1 can record, as the load fluctuation information, the fluctuation of the load value detected by load sensor 50 multiple times during the series of operations of assistance device 10, or continuously for a certain period.

The application information is used to grasp applications for which user H used assistance device 10. In the example illustrated in FIG. 6, the application information is action record information on actions performed by user H during use of the assistance device. As a result, the caregiver or the like can grasp the applications for which the care receiver who is user H uses assistance device 10. In usage record data M1 illustrated in FIG. 6, an action type code assigned according to an action content is recorded as action record information. In the present embodiment, the action record information is information that cannot be acquired from assistance device 10, and is recorded in management device 100 by the caregiver or the like.

It should be noted that usage record data M1 may include, as the application information, usage location information as to the location at which assistance device 10 is used. For example, assistance system 1 can mount a GPS transmitter on assistance device 10, and acquire the information transmitted from the GPS transmitter during the operation of assistance device 10 as the usage location information. In this case, since assistance system 1 can obtain the application information from assistance device 10, it is possible to omit a recording operation of the application information by the caregiver or the like.

### 7. Specific Example of Assistance Record Data M2

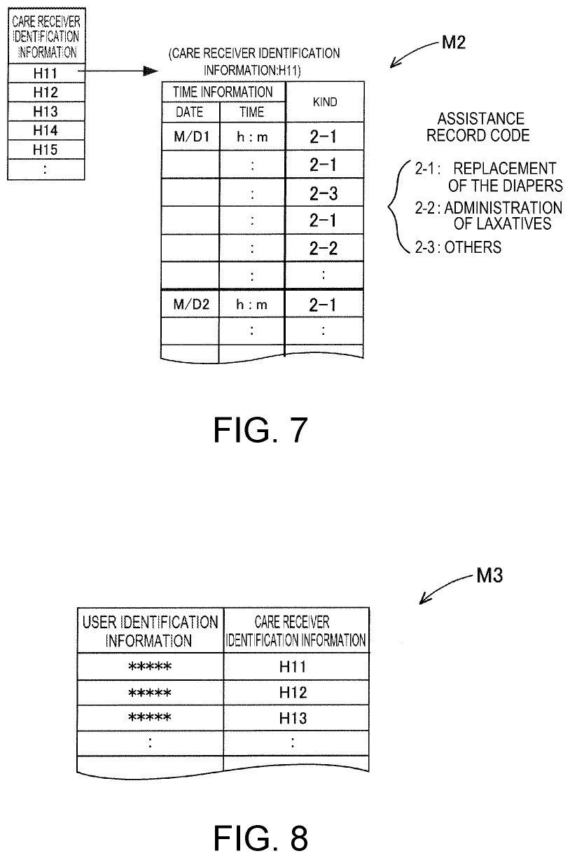

Next, a specific example of assistance record data M2 will be described with reference to FIG. 7. In assistance record data M2 illustrated in FIG. 7, assistance record information by the care receiver identification information is recorded in association with the assistance time information.

In an example illustrated in FIG. 7, the care receiver identification information is information associated with the user identification information as described above. In the example illustrated in FIG. 7, ID information (H11, H12, . . .) assigned to each of the care receiver who is user H is recorded as the care receiver identification information. The time information is assistance date and time information as to a date and time when the assistance was provided to the care receiver. The assistance record information is record information as to assistance that does not involve the use of assistance device 10, is record information that cannot be acquired from assistance device 10, and an assistance type code assigned in accordance with an assistance content is recorded in assistance record data M2 illustrated in FIG. 7. Examples of the content of assistance recorded as the assistance record information include replacement of diapers, administration of laxatives, and the like.

### 8. Concrete Example of Care Receiver Data M3

Next, a specific example of care receiver data M3 will be described with reference to FIG. 8. Care receiver data M3 stores care receiver identification information as to the care receiver registered in advance as user H of assistance device 10 in association with the user identification information. That is, the user identification information included in usage record data M1 and the care receiver identification information included in assistance record data M2 are associated with each other via care receiver data M3. It should be noted that the care receiver identification information may be the same as the user identification information.

### 9. Flow of Information Extraction by Extracting Section 103

Next, a flow of information extraction by extracting section 103 will be described with reference to a specific example. As illustrated in FIG. 6 to FIG. 8, when input section 102 receives the input of the care receiver identification information, extracting section 103 collates the inputted care receiver identification information with care receiver data M3. Then, extracting section 103 extracts the user identification information associated with the corresponding care receiver identification information from care receiver data M3.

Next, extracting section 103 collates the extracted user identification information with usage record data M1. Then, extracting section 103 extracts the usage information and the usage time information associated with the extracted user identification information. Similarly, extracting section 103 collates the care receiver identification information with assistance record data M2, and extracts the care receiver record information and the assisted time information associated with the corresponding care receiver identification information. In this manner, extracting section 103 extracts information on the care receiver registered as user H from usage record data M1 and assistance record data M2 stored in storage section 101.

### 10. Specific Examples of Display Data

Next, specific examples of the display data will be described with reference to FIGS. 9 to 13. Display data generating section 105 generates display data in which various types of information extracted by extracting section 103 are processed in a graspable manner the time series change in the usage information. FIGS. 9 to 13 illustrate examples of display data displayed by display section 104 on display device 104a.

FIG. 9 illustrates, as display data 105a indicating a time series change of the usage information, a graph indicating a transition of the usage frequency information as to the number of uses of assistance device 10 per a predetermined unit period. In an example illustrated in FIG. 9, display data generating section 105 calculates the number of uses of assistance device 10 for each predetermined period based on the date information included in the time information, and generates display data 105a based on the calculation result. For example, extracting section 103 extracts usage information and time information for four weeks as to specific user H from usage record data M1. Then, display data generating section 105 calculates the number of uses of assistance device 10 per week in the four weeks, and generates display data 105a illustrated in FIG. 9.

The caregiver or the like can predict a change in the physical condition of the care receiver who is user H by referring to the graph indicating the transition of the usage frequency information of assistance device 10. That is, display data 105a can provide a trigger for the caregiver or the like to realize that the physical condition of user H tends to recover or deteriorate when there is an increase or decrease in the number of uses of assistance device 10 with respect to specific user H.

FIG. 10 illustrates a graph illustrating a transition of specific load value information as display data 105b indicating time series changes of usage information. In the example illustrated in FIG. 10, extracting section 103 extracts the usage time information and the specific load value information as to specific user H from usage record data M1. Subsequently, display data generating section 105 generates display data 105b exemplified in FIG. 10 as a display graph indicating the transition of the specific load value information based on the usage time information and the specific load value information. It should be noted that the specific load value information is, for example, a load value detected by load sensor 50 at a specific timing, and exemplified by a load value or the like when assistance device 10 in operation is in a specific posture (e.g., a posture or the like of assistance device 10 in which user H is in the stand-up posture).

The caregiver or the like can predict a change in the physical condition of the care receiver who is user H by referring to the graph indicating the transition of the specific load value information. That is, display data 105b can provide a trigger for the caregiver or the like to realize that the physical condition of specific user H tends to recover or deteriorate when there is an increase or decrease in the specific load value information with respect to specific user H. Specifically, when the specific load value information tends to increase, the caregiver or the like can recognize that the muscle strength of the lower body of the care receiver who is user H may be reduced. In addition, when the specific load value information tends to decrease, the caregiver or the like can recognize that the muscle strength of the lower body of the care receiver who is user H tends to recover.

In addition, display section 104 can simultaneously display on display device 104a both display data 105a indicating the transition of the usage frequency information illustrated in FIG. 9 and display data 105b indicating the transition of the load value information illustrated in FIG. 10. At this time, display section 104 can display, on display device 104, display data 105a indicating the transition of the usage frequency information and display data 105b indicating the transition of the load value information in a state in which units of the horizontal axis (time axis) of display data 105a and display data 105b are the same. As a result, the caregiver or the like can easily grasp, for example, whether or not the increase or decrease of the usage frequency information and the increase or decrease of the load value information are linked with each other.

Specifically, as long as the specific load value tends to increase with respect to specific user H and the number of uses of assistance device 10 tends to decrease, the caregiver or the like can recognize that the physical condition of the care receiver who is user H tends to deteriorate. Similarly, if the load value tends to increase with respect to specific user H and the number of uses thereof tends to increase, the caregiver or the like can recognize that the physical condition of the care receiver who is user H tends to recover.

As described above, display section 104 can simultaneously arrange and display the multiple graphs generated by display data generating section 105 side by side on display device 104a. Then, by displaying multiple types of information as to the use status of specific user H simultaneously side by side on display device 104a, management device 100 can provide a trigger for the caregiver or the like to recognize a change in the physical condition of user H at an early stage.

FIG. 11 illustrates an example of a graph indicating the transition of load fluctuation information of assistance device 10 as display data 105c indicating the time series change in the usage information. In the example illustrated in FIG. 11, extracting section 103 extracts multiple pieces of load value information when specific user H uses assistance device 10 at a specific timing from usage record data M1. At this time, for example, extracting section 103 extracts multiple pieces of load value information when assistance device 10 is used (for example, when assistance device 10 is first used at the predetermined date) at a predetermined timing of a predetermined date within a predetermined period (for example, the first Monday of each month, the 10th of each month, or the like). Next, display data generating section 105 generates display data 105c including multiple graphs indicating the transition of the load fluctuation information illustrated in FIG. 11, based on the extracted usage time information and the load value information.

Specifically, in the example illustrated in FIG. 11, extracting section 103 extracts the load value information six times detected by load sensor 50 at different timings during a series of operations of assistance device 10. Then, display data generating section 105 generates a graph in which the extracted load value information for six times is arranged in time series in the horizontal axis direction, for each of the extracted usage time information. In the example illustrated in FIG. 11, load value number 1 is the load value information detected by load sensor 50 immediately after the transition from the ready-to-stand state to the standing state is initiated, and load value number 6 is the load value detected by load sensor 50 in the standing completion state. In the example illustrated in FIG. 11, display data 105c displays multiple graphs generated by time information in an overlapping manner. As a result, the caregiver or the like can easily grasp the transition of the load fluctuation information with the passage of time.

The caregiver or the like can predict a change in the physical condition of the care receiver, who is specific user H, by referring to the transition of the load fluctuation information. That is, display data 105c can provide a trigger for the caregiver or the like to realize that the physical condition of the care receiver who is specific user H tends to recover or deteriorate when there is a fluctuation in the load fluctuation information with respect to specific user H. For example, when a tendency is seen that a timing at which the load value increases is earlier with the passage of time, the caregiver or the like can recognize that muscle strength of the lower body of the care receiver who is user H may be reduced. In addition, when a tendency is seen that the timing at which the load value increases is delayed with the lapse of time, the caregiver or the like can recognize that the muscle strength of the lower body of the care receiver who is user H may be recovered.

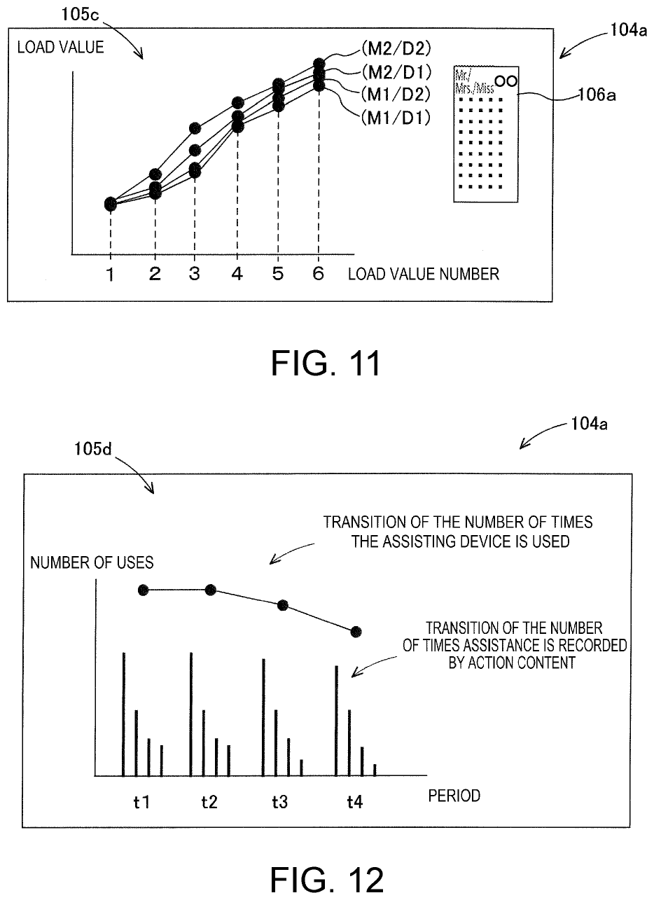

FIG. 12 illustrates an example of a graph collectively illustrating a transition of the number of uses of assistance device 10 and a transition of the number of action records by action content, as display data 105d indicating the time series change in the usage information.

In the example illustrated in FIG. 12, extracting section 103 extracts usage time information as to specific user H from usage record data M1. Next, display data generating section 105 calculates the number of uses of assistance device 10 for each predetermined period based on the date information included in the extracted usage time information, and generates display data 105d indicating the transition of the usage frequency information of assistance device 10.

In addition, extracting section 103 extracts the assistance time information and the assistance record information from assistance record data M2 of specific user H (the care receiver) based on the care receiver identification information associated with the user identification information held by specific user H. It should be noted that the assistance record information extracted from assistance record data M2 and the usage information extracted from usage record data M1 have the same date information included in the usage time information or assistance time information associated with them respectively. Subsequently, display data generating section 105 calculates the number of assistance records by assistance content for each predetermined period based on the date information and the assistance record information included in the extracted assistance time information, and generates a graph indicating a transition of the assistance record frequency information by assistance content.

Then, display data generating section 105 generates display data 105d exemplified in FIG. 12 by superimposing a graph indicating the transition of the usage frequency information of assistance device 10 and a graph indicating the transition of the assistance record frequency information by assistance content. As described above, display section 104 displays the time series change in the assistance record information in a manner that can be compared with the time series change in the usage information.

By referring to display data 105d illustrated in FIG. 12, the caregiver or the like can easily compare the number of uses of assistance device 10 with the number of actions by action content. For example, when there is an increase or decrease in the number of uses of assistance device 10 with respect to specific user H, the caregiver or the like can assume a factor of the increase or decrease in the number of uses by referring to the increase or decrease in the number of actions by action content.

In addition, the caregiver or the like can perform the effect measurement due to the care receiver's use of assistance device 10 based on the number of uses of assistance device 10 and the number of actions by action content. For example, the caregiver or the like can grasp an effect that the use of assistance device 10 has on the physical condition of the care receiver in case in multiple users H, a tendency is seen that the increase or decrease of the number of uses of assistance device 10 and the increase or decrease of the specific action content is linked with each other.

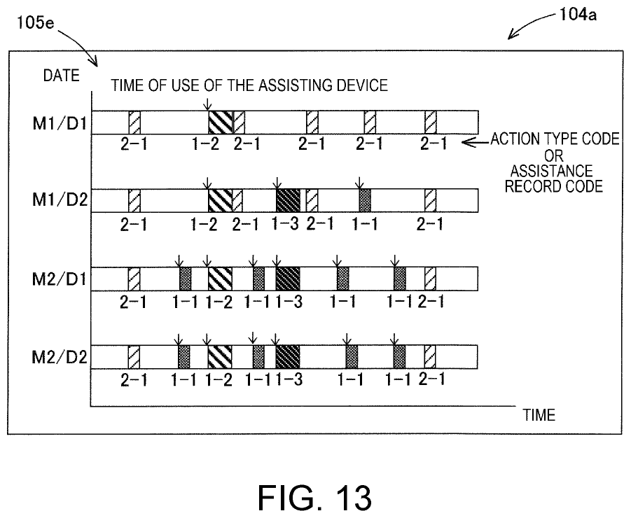

FIG. 13 illustrates, as display data 105e indicating the time series change in the usage information, an example of a graph indicating a relationship between the application information of user H of assistance device 10 and the assistance record information for the care receiver who is user H, and the time information when the user used assistance device 10.

In an example illustrated in FIG. 13, extracting section 103 extracts, from usage record data M1, application information for multiple determined dates within a predetermined period. Next, display data generating section 105 generates a graph in which the extracted application information is displayed in the time series order for each extracted date. Further, extracting section 103 extracts, from assistance record data M2, the assistance record information at multiple determined dates within a predetermined period. It should be noted that the assistance record information extracted from assistance record data M2 and the application information extracted from usage record data M1 have the same date information included in the usage time information or assistance time information associated with them respectively. Next, display data generating section 105, while arranging the extracted assistance record information in time series, adds it to the graph indicating the application information, and generates display data 105e as illustrated in FIG. 13.

By referring to display data 105e illustrated in FIG. 13, the caregiver or the like can grasp an intended application of assistance device 10 by specific user H and the number of uses of assistance device 10 per day. In addition, the caregiver or the like can easily compare the increase or decrease of the usage and the number of uses of assistance device 10 with the transition of the assistance record. In this case, when there is a change in the assistance record with respect to a specific care receiver, the caregiver or the like can find a causal relationship between the change in the assistance record and the application for use and the number of uses of assistance device 10. That is, the caregiver or the like can assume the effect of the use of assistance device 10 on the physical condition of the care receiver.

### 11. Analysis by Analysis Section 106

Next, analysis by analysis section 106 will be described. Analysis section 106 analyzes an assumed change in the physical condition of user H based on the transition of the number of uses of assistance device 10, the transition of the load value information, the transition of the load fluctuation information, and the like. Then, display section 104 displays analysis result 106a by analysis section 106 on display device 104a.

For example, analysis section 106 can list, as analysis result 106a, items assumed to be a factor causing a decrease in the number of uses of assistance device 10 based on a transition of the usage frequency information of assistance device 10 illustrated in FIG. 9. Examples of the “items assumed to be a factor causing a decrease in the number of uses” include: a decrease in action desire due to muscle weakness in the lower body of the care receiver; a recovery of the muscle strength in the lower body of the care receiver, making it possible for the care receiver to act without using assistance device 10, and the like.

In this manner, management device 100 can notify the caregiver or the like that there is a sign of a change in the physical condition of user H by displaying analysis result 106a by analysis section 106. As a result, the caregiver or the like can quickly grasp the change in the physical condition of user H.

In the present embodiment, although display section 104 has been described as an example in which both display data 105a to 105e generated by display data generating section 105 and analysis result 106a by analysis section 106 are displayed on display device 104a, the present invention is not limited to this. That is, display section 104 can display only either one of display data 105a to 105e and analysis result 106a on display device 104a.

In addition, analysis section 106 can notify that the physical condition of the care receiver who is user H may be deteriorated when there is a sudden change (e.g., a sudden decrease in the number of uses) in the transition of the usage frequency information of assistance device 10. For example, analysis section 106 may periodically perform analysis to display a list of users H who have no history of using assistance device 10 within a predetermined time period. In this case, the caregiver or the like can detect an abrupt change in the physical condition of the care receiver at an early stage based on the notification or the list display by analysis section 106.

### 12. Variant of the Embodiment

In the embodiment, management device 100 acquires usage record data M1 recorded by assistance device 10 via management server 90. On the other hand, management device 100 may obtain usage record data M1, for example, by directly communicating with assistance device 10 via the LAN. In addition, assistance system 1 may configure a part of the configuration of management device 100 as assistance device 10 or management server 90. For example, assistance system 1 may be configured to generate display data in assistance device 10 or management server 90 and to store the generated display data in storage section 101 of management device 100.

### REFERENCE SIGNS LIST

1: Assistance system, 10: Assistance device, 50: Load sensor, 101: Storage section, 103: Extracting section, 104: Display section, 105a-105e: Display data, 106a: Analysis result, H: User of assistance device, M1: Usage record data, M2: Assistance record data

1. An assistance system, comprising:
a storage section configured to store usage record data in which usage information as to a use of an assistance device configured to assist an operation of a care receiver is recorded in association with time information as to a date and time when the assistance device is used and user identification information used to identify a user using the assistance device;
an extracting section configured to extract the usage information and the time information as to a specific user based on the user identification information included in the usage record data; and
a display section configured to display a time series change in the usage information as to the specific user in a graspable manner.

2. The assistance system according to claim 1, wherein the usage information includes usage frequency information as to the number of uses of the assistance device per predetermined unit period, and the display section displays a transition of the usage frequency information as the time series change of the usage information.

3. The assistance system according to claim 1, wherein the usage information includes load value information as to a load value received by the assistance device when the assistance device is used, and the display section displays a transition of the load value information as the time series change of the usage information.

4. The assistance system according to claim 3, wherein the load value information is the load value received when the assistance device is in a specific posture.

5. The assistance system according to claim 1, wherein the usage information includes load fluctuation information as to a fluctuation in the load value received by the assistance device during an operation of the assistance device, and the display section is configured to display a transition of the load fluctuation information as the time series change of the usage information.

6. The assistance system according to claim 3, wherein the assistance device includes a load sensor capable of detecting the load value received from a portion supporting an upper body of the user, and the load value is a detection value detected by the load sensor.

7. The assistance system according to claim 1, wherein the usage information includes application information capable of grasping an application of the assistance device.

8. The assistance system according to claim 7, wherein the application information includes action record information as to an action performed by the user while using the assistance device.

9. The assistance system of claim 7, wherein the application information includes usage location information as to a location at which the assistance device is used.

10. The assistance system according to claim 1, wherein the display section presents an analysis result as to a change in a physical condition of the user based on the time series change in the usage information.

11. The assistance system according to claim 1, wherein the storage section is configured to store assistance record data in which assistance record information, which is information as to an assistance performed without using the assistance device to the care receiver who is the user, is recorded in association with the time information and the user identification information; the extracting section is configured to extract the assistance record information and the time information as to a specific care receiver based on the user identification information included in the assistance record data; and the display section is configured to display the assistance record information as to the specific care receiver in a manner that can be compared with the time series change in the usage information.

12. An assistance system, comprising:
a storage section configured to store usage record data in which usage information as to a use of an assistance device configured to assist an operation of a care receiver is recorded in association with time information as to a date and time when the assistance device is used and user identification information used to identify a user using the assistance device;
an extracting section configured to extract the usage information and the time information as to a specific user based on the user identification information included in the usage record data; and
a display section configured to display an analysis result as to a change in a physical condition of the user based on a time series change in the usage information as to the specific user.

13. The assistance system according to claim 2, wherein the usage information includes load value information as to a load value received by the assistance device when the assistance device is used, and the display section displays a transition of the load value information as the time series change of the usage information.

14. The assistance system according to claim 4, wherein the assistance device includes a load sensor capable of detecting the load value received from a portion supporting an upper body of the user, and the load value is a detection value detected by the load sensor.

15. The assistance system according to claim 5, wherein the assistance device includes a load sensor capable of detecting the load value received from a portion supporting an upper body of the user, and the load value is a detection value detected by the load sensor.

16. The assistance system according to claim 2, wherein the display section presents an analysis result as to a change in a physical condition of the user based on the time series change in the usage information.

17. The assistance system according to claim 3, wherein the display section presents an analysis result as to a change in a physical condition of the user based on the time series change in the usage information.

18. The assistance system according to claim 4, wherein the display section presents an analysis result as to a change in a physical condition of the user based on the time series change in the usage information.

19. The assistance system according to claim 2, wherein the storage section is configured to store assistance record data in which assistance record information, which is information as to an assistance performed without using the assistance device to the care receiver who is the user, is recorded in association with the time information and the user identification information; the extracting section is configured to extract the assistance record information and the time information as to a specific care receiver based on the user identification information included in the assistance record data; and the display section is configured to display the assistance record information as to the specific care receiver in a manner that can be compared with the time series change in the usage information.

20. The assistance system according to claim 3, wherein the storage section is configured to store assistance record data in which assistance record information, which is information as to an assistance performed without using the assistance device to the care receiver who is the user, is recorded in association with the time information and the user identification information; the extracting section is configured to extract the assistance record information and the time information as to a specific care receiver based on the user identification information included in the assistance record data; and the display section is configured to display the assistance record information as to the specific care receiver in a manner that can be compared with the time series change in the usage information.
